(19) United States
(12) Patent Application Publication
(10) Pub. No.: US 2025/0282767 A1
(43) Pub. Date: Sep. 11, 2025

(54) PIKFYVE KINASE INHIBITORS

(71) Applicant: AcuraStem Incorporated, Pasadena, CA (US)

(72) Inventor: Kirsten BJERGARDE, Pasadena, CA (US)

(21) Appl. No.: 18/862,354

(22) PCT Filed: Apr. 25, 2023
(86) PCT No.: PCT/US2023/019770

§ 371 Date: Nov. 1, 2024

Related U.S. Application Data
(60) Provisional application No. 63/363,962, filed on May 2, 2022.

(51) Int. Cl. C07D 413/14; A61K 31/5377

(52) U.S. Cl. CPC C07D 413/14; A61K 31/5377

(57) ABSTRACT

The present invention relates to compounds of formula (I) useful as inhibitors of phosphatidylinositol-3-phosphate 5-kinase (PIKfyve) as well as their use for treating diseases and disorders associated with PIKfyve.

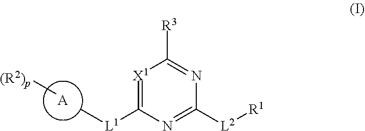

### CROSS-REFERENCE TO RELATED APPLICATIONS

The present application claims priority to U.S. Provisional Application No. 63/363,962 filed May 2, 2022. The aforementioned application is hereby incorporated by reference in its entirety.

### FIELD OF THE INVENTION

The present invention relates to compounds useful as inhibitors of phosphatidylinositol-3-phosphate 5-kinase (PIKfyve).

### BACKGROUND OF THE INVENTION

Apilimod is recognized as a potent transcriptional inhibitor of IL-12 and IL-23. See Wada et al., Blood 109 (2007):1156-1164. IL-12 and IL-23 are inflammatory cytokines normally produced by immune cells, such as B-cells and macrophages, in response to antigenic stimulation. Autoimmune disorders and other disorders characterized by chronic inflammation are characterized in part by inappropriate production of these cytokines. In immune cells, the selective inhibition of IL-12/IL-23 transcription by apilimod was recently shown to be mediated by apilimod's direct binding to phosphatidylinositol-3-phosphate 5-kinase (PIKfyve). See Cai et al., Chemistry and Biol. 20 (2013):912-921. PIKfyve plays a role in Toll-like receptor signaling, which is important in innate immunity.

Based upon its activity as an immunomodulatory agent and a specific inhibitor of IL-12/IL-23, apilimod has been proposed as useful in treating autoimmune and inflammatory diseases and disorders. See U.S. Pat. Nos. 6,858,606 and 6,660,733 (describing a family of pyrimidine compounds, including apilimod, purportedly useful for treating diseases and disorders characterized by IL-12 or IL-23 overproduction, such as rheumatoid arthritis, sepsis, Crohn's disease, multiple sclerosis, psoriasis, or insulin dependent diabetes mellitus). Similarly, apilimod was suggested to be useful for treating certain cancers based upon its activity to inhibit c-Rel or IL-12/23, particularly in cancers where these cytokines were believed to play a role in promoting aberrant cell proliferation. See WO 2006/128129 and Baird et al., Frontiers in Oncology 3:1 (2013, respectively). International Publication Nos. WO 2016/210372 and WO 2019/046316 and U.S. Patent Publication No. 2018/0161335, which are hereby incorporated by reference, disclose methods of treating a neurological disease such as amyotrophic lateral sclerosis with a PIKfyve kinase inhibitor.

### SUMMARY OF THE INVENTION

In one aspect, the present invention is directed to a compound of the formula (I):

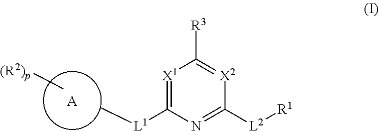

or a pharmaceutically acceptable salt thereof, wherein
R¹ is hydroxy, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;
each occurrence of R² is independently substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl;
R³ a 5- or 6-membered oxygen containing moiety, such as a moiety selected from

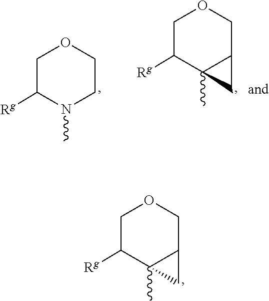

wherein each R^{g} is, independently, selected from H, substituted or unsubstituted alkyl (e.g., methyl or ethyl), OH, substituted or unsubstituted alkoxy (e.g., methoxy), halogen, CN and —NHR^{m} (where R^{m} is H or substituted or unsubstituted alkyl (e.g., methyl or ethyl));
Ring A is (i) a 5 or 6-membered heteroaryl, a 5-6, 6-5 or 6-6 membered bicyclic heteroaryl, or a heterocyclyl, each having at least one nitrogen or oxygen ring atom, or (ii) phenyl;
L¹ is absent, C₁-C₂ alkylene, —NR^{c}—, —O—, —S—, —C(O)—, —NHC(O)—, —C(O)NH—, —NR^{c}C(O)—, or —NR^{c}C(O)(CR^{a}R^{b})ₘ—;
L² is absent, —O—, —O—(CR^{a}R^{b})ₘ—, —(CR^{a}R^{b})ₘ—, —NR^{c}—(CR^{a}R^{b})ₘ—, or —S—(CR^{a}R^{b})ₘ—;
(i) X¹ is CH or CR^{c} and X² is N, or (ii) X¹ is N and X² is N, CH, or CR^{c};
each occurrence of R^{a} and R^{b} is independently hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e};
each occurrence of R^{c} is independently a hydrogen or C₁₋₆ alkyl (e.g., C₁-C₄ alkyl);
each occurrence of R^{d} and R^{e} is independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;
each occurrence of m is independently 1-4; and
p is 1 or 2.

In one preferred embodiment, R³ is selected from

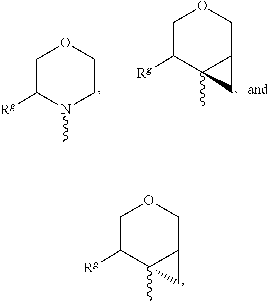

wherein each R^{g} is, independently, selected from H, substituted or unsubstituted alkyl (e.g., methyl or ethyl), OH, substituted or unsubstituted alkoxy (e.g., methoxy or ethoxy), halogen, CN and —NHR^{m} (where R^{m} is H or substituted or unsubstituted alkyl (e.g., methyl or ethyl)). In one preferred embodiment, when R³ is

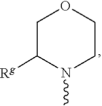

R^{g} is not hydrogen.

In one embodiment, X¹ is CH, X² is N, L¹ is absent, and L² is absent.

In one embodiment, the compound of formula (I) possesses at least one of:
(i) L¹ is —NR^{c}C(O)— or —NR^{c}C(O)(CR^{a}R^{b})ₘ—,
(ii) L² is —O—, or
(iii) X² is CH or CR^{c}.

These compounds are useful as PIKfyve kinase inhibitors.

In one embodiment, R¹ is heterocyclyl or heteroaryl. For example, R¹ may be selected from (the squiggly lines indicate the point of attached to the rest of the molecule)

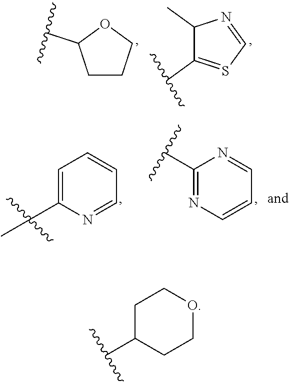

In another embodiment, R¹ is hydroxy.

In yet another embodiment, when L² is absent, R¹ is selected from

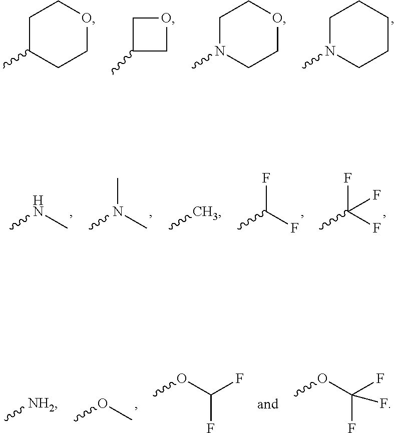

In yet another embodiment, when L² is present (not absent), R¹ is selected from

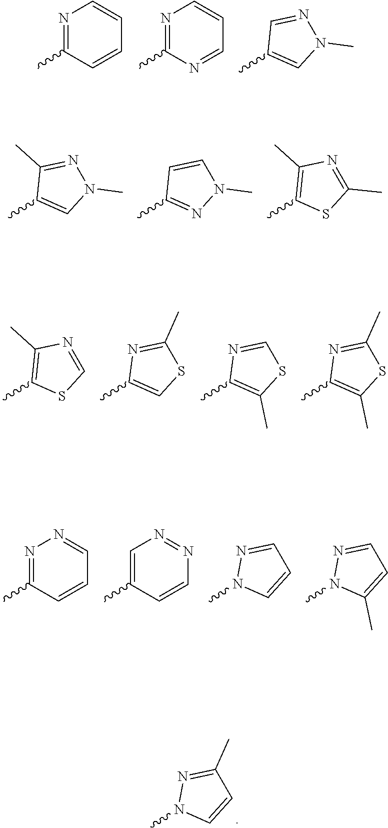

In one embodiment of any of the compounds described herein, each occurrence of R² is independently substituted or unsubstituted heteroaryl, such as, for example, substituted or unsubstituted indolyl, substituted or unsubstituted indazolyl, substituted or unsubstituted 2,2-dimethylindolyl, substituted or unsubstituted indolin-2-one, substituted or unsubstituted 1,3-dihydro-2H-benzo[d]imidazole-2-one, substituted or unsubstituted benzo[b]thiophenyl, substituted or unsubstituted pyrrolo[3,2-b]pyridinyl, substituted or unsubstituted pyrrolo[2,3-b]pyridinyl, or substituted or unsubstituted thieno[2,3-b]pyridinyl.

In one embodiment of any of the compounds described herein, each occurrence of R² is independently selected from

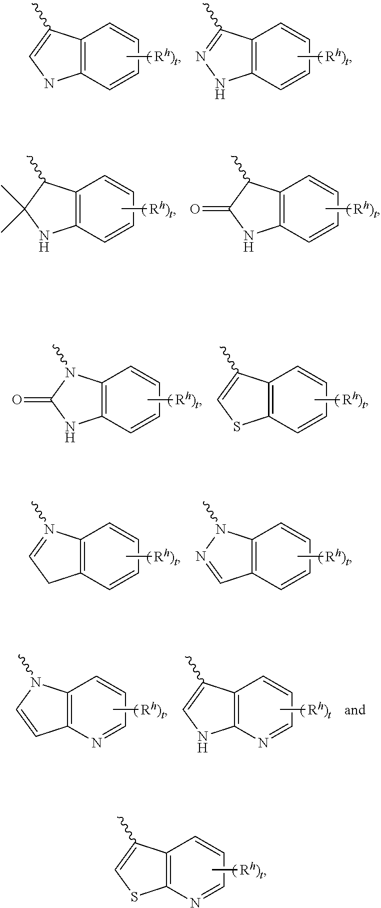

wherein
each occurrence of R^{h} is, independently H, halogen, substituted or unsubstituted alkyl (e.g., methyl or ethyl), substituted or unsubstituted alkoxy (e.g., methoxy or ethoxy), —CN, —NH₂, —NHR^{m} or —NR^{m}₂, or two R^{h} are joined to form a saturated or unsaturated 5- to 7-membered ring;
each R^{m} is, independently, R^{m} is H or substituted or unsubstituted alkyl (e.g., methyl or ethyl); and
t is 0, 1, or 2.

In one embodiment, each occurrence of R² is independently substituted or unsubstituted aryl, such as a substituted or unsubstituted phenyl. For instance, R² may be phenyl, a halogen-substituted phenyl, an alkyl-substituted phenyl (e.g., a C₁₋₄ alkyl-substituted phenyl), a halogenated alkyl-substituted phenyl, or an alkoxy-substituted phenyl. In one embodiment, R² is selected from phenyl, 3-methoxyphenyl, 3-methylphenyl, 3-trifluoromethylphenyl, and 3-chlorophenyl. In an embodiment, R² is selected from phenyl, 3-methoxyphenyl, and 3-methylphenyl.

In another embodiment, each occurrence of R² is independently substituted or unsubstituted alkyl (such as a C₁₋₄ alkyl). For instance, R² can be unsubstituted isopropyl.

In one embodiment, R³ is a substituted or unsubstituted, saturated or unsaturated nitrogen- or oxygen-containing heterocyclyl. For instance, R³ can be a substituted or unsubstituted, saturated or unsaturated 5-10 membered (such as a 5-8 membered) mono- or bi-cyclic heterocyclyl having at least one nitrogen or oxygen ring atom. In one embodiment, R³ is a substituted or unsubstituted 5-10 membered (such as a 5-8 membered) mono- or bi-cyclic heterocyclyl having at least one nitrogen atom and optionally an oxygen ring atom, where the nitrogen ring atom is directly attached to the rest of the molecule. In one embodiment, R³ is a substituted or unsubstituted (unsaturated) 5-membered monocyclic heterocyclyl having an oxygen ring atom or a nitrogen ring atom.

In another embodiment, R³ is a substituted or unsubstituted, saturated or unsaturated 6-membered monocyclic heterocyclyl having an oxygen ring atom and optionally a nitrogen ring atom. In yet another embodiment, R³ is a saturated 8-membered bicyclic heterocyclyl having a nitrogen ring atom and an oxygen ring atom.

In one embodiment of any of the compounds described herein, R³ is selected from

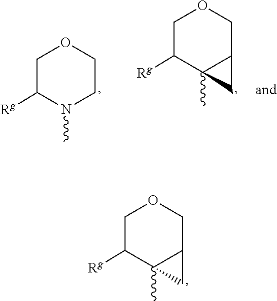

wherein each R^{g} is, independently, selected from H, substituted or unsubstituted alkyl (e.g., methyl or ethyl), OH, substituted or unsubstituted alkoxy (e.g., methoxy), halogen, CN and —NHR^{m} where R^{m} is H or substituted or unsubstituted alkyl (e.g., methyl or ethyl), with the proviso that R³ is not unsubstituted morpholinyl.

In one embodiment of any of the compounds described herein, ring A is a 5-membered heteroaryl having at least one nitrogen ring atom. In one embodiment of any of the compounds described herein, ring A includes two heteroatoms as ring atoms (such as two nitrogen ring atoms, or one nitrogen ring atom with one sulfur ring atom).

In one embodiment of any of the compounds described herein (where p is 1), ring A is selected from

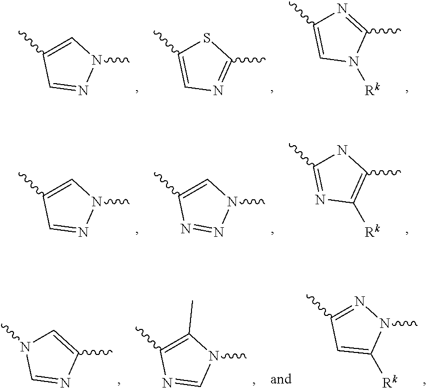

wherein
each R^{k} is, independently, hydrogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl, and
each squiggly line ( [symbol] ) independently represents the point of attachment of ring A to variable L¹ or R² to ring A (preferably, the righthand squiggly line in the ring A groups is attached to L¹ (preferably where L¹ is absent), and the left-hand squiggly line is attached to R²).

In one embodiment, the righthand squiggly line in the ring A groups is attached to L¹ (preferably where L¹ is absent), and the left-hand squiggly line is attached to R². In another embodiment, L¹ is absent.

In another embodiment of any of the compounds described herein, ring A is selected from

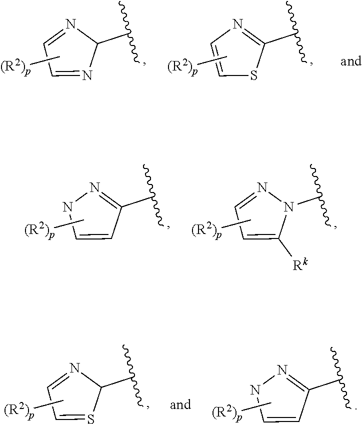

For instance, ring A can be selected from

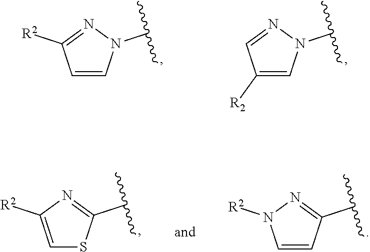

In one embodiment of any of the compounds described herein, the R² group in any ring A above is selected from substituted or unsubstituted aryl, such as a substituted or unsubstituted phenyl. For instance, R² may be phenyl, an alkyl-substituted, or an alkoxy-substituted phenyl. In an embodiment of any of the compounds described herein, R² is selected from phenyl, 3-methoxyphenyl, and 3-methylphenyl.

In one embodiment of any of the compounds described herein, ring A is a heterocyclyl having at least one oxygen ring atom. In one embodiment of any of the compounds described herein, the heterocyclyl is a lactone.

In one embodiment of any of the compounds described herein, ring A is a heterocyclyl having at least one nitrogen ring atom. In one embodiment of any of the compounds described herein, the heterocyclyl is a lactam. In certain embodiments, the lactam is a 5-membered lactam.

In one embodiment of any of the compounds described herein, L¹ is absent.

In another embodiment of any of the compounds described herein, L¹ is —NH—, —N(CH₃)—, —O—, or —CH₂—. In one embodiment of any of the compounds described herein, L¹ is —NH—. In another embodiment of any of the compounds described herein, L¹ is —C(O)NH— (where the carbonyl is attached to the rest of the molecule and the nitrogen is attached to ring A). In yet another embodiment of any of the compounds described herein, L¹ is —NHC(O)— (where the nitrogen atom is attached to the rest of the molecule and the carbonyl is attached to ring A).

In one embodiment of any of the compounds described herein, L² is —O—(CR^{a}R^{b})ₘ—. In one embodiment of any of the compounds described herein, L² is —OCH₂CH₂— or —OCH₂—. In another embodiment, of any of the compounds described herein L² is —OCH₂CH₂CH(OH)CH₂—.

In another embodiment of any of the compounds described herein, L² is —(CR^{a}R^{b})ₘ—. In one embodiment of any of the compounds described herein, L² is —CH₂CH₂—.

In yet another embodiment of any of the compounds described herein, L² is —NR^{c}—(CR^{a}R^{b})ₘ—, such as —NH—(CR^{a}R^{b})ₘ— (e.g., —NH—, —NHCH₂—, and —NHCH₂CH₂—).

In one embodiment of any of the compounds described herein, -L²-R¹ is —OCH₂CH₂CH(OH)CH₂OH.

In one embodiment of any of the compounds described herein, L² is —O—.

In one embodiment of any of the compounds described herein, X¹ is CH. In another embodiment, X¹ is N.

In one embodiment of any of the compounds described herein, each occurrence of R^{a} and R^{b} is independently hydrogen, hydroxy, or hydroxy(C₁₋₄)alkyl. In another embodiment, each occurrence of R^{a} and R^{b} is independently hydrogen or hydroxy.

In one embodiment of any of the compounds described herein, m is 1. In another embodiment of any of the compounds described herein, m is 2. In a embodiment of any of the compounds described herein, m is 1 or 2 when R¹ is cyclic. In another embodiment of any of the compounds described herein, m is 3 or 4 when R¹ is acyclic.

In an embodiment of any of the compounds described herein, p is 1.

In another embodiment of any of the compounds described herein, p is 2.

In one embodiment of any of the compounds described herein, the moiety

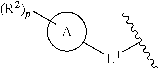

is selected from

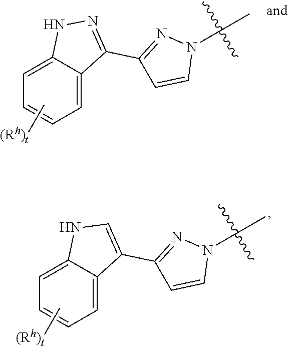

where
each occurrence of R^{h} is, independently H, halogen, substituted or unsubstituted alkyl (e.g., methyl or ethyl), substituted or unsubstituted alkoxy (e.g., methoxy or ethoxy), —CN, —NH₂, —NHR^{m} or —NR^{m}₂, or two R^{h} are joined to form a saturated or unsaturated 5- to 7-membered ring;
each R^{m} is, independently, R^{m} is H or substituted or unsubstituted alkyl (e.g., methyl or ethyl); and
t is 0, 1, or 2.
   the variables R^{h} and t are as defined herein.

In one aspect, the present invention is directed to a compound of formula (I).

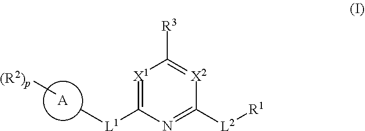

or a pharmaceutically acceptable salt thereof, wherein
R¹ is selected from

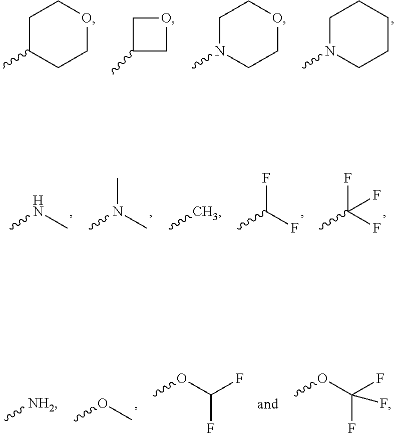

each occurrence of R² is independently selected from

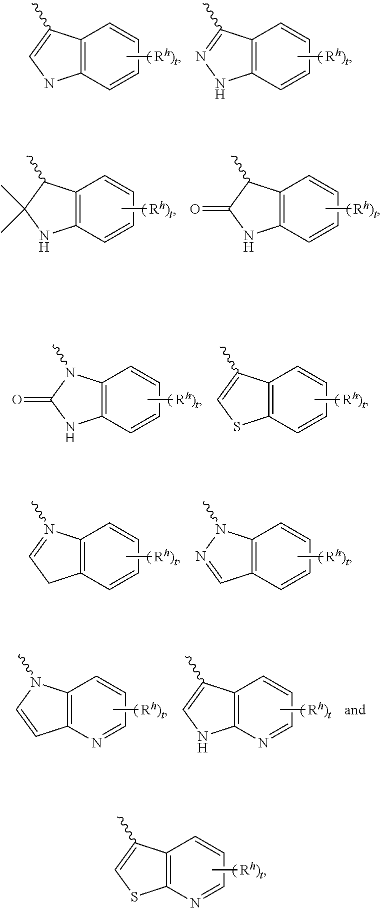

each occurrence of R^{h} is, independently H, halogen, substituted or unsubstituted alkyl (e.g., methyl or ethyl), substituted or unsubstituted alkoxy (e.g., methoxy or ethoxy), —CN, —NH₂, —NHR^{m} or —NR^{m}₂, or two R^{h} are joined to form a saturated or unsaturated 5- to 7-membered ring;
each R^{m} is, independently, H or substituted or unsubstituted alkyl (e.g., methyl or ethyl);
t is 0, 1, or 2;
R³ is selected from

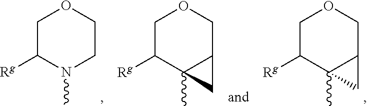

each R^{g} is, independently, selected from H, substituted or unsubstituted alkyl (e.g., methyl or ethyl), OH, substituted or unsubstituted alkoxy (e.g., methoxy), halogen, —CN and —NHR^{m} (where R^{m} is H or substituted or unsubstituted alkyl (e.g., methyl or ethyl));
Ring A is selected from

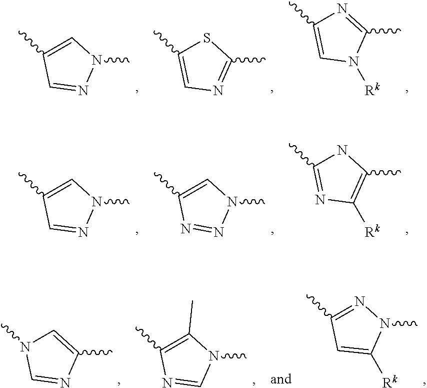

where
each R^{k} is, independently, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl, and
each squiggly line ( [symbol] ) independently represents the point of attachment of ring A to variable L¹ or R² to ring A (preferably, the righthand squiggly line in the ring A groups is attached to L¹ (preferably where L¹ is absent), and the left-hand squiggly line is attached to R²);
L¹ is absent, C₁-C₂ alkylene, —NR^{c}—, —O—, —S—, —C(O)—, —NHC(O)—, —C(O)NH—, —NR^{c}C(O)—, or —NR^{c}C(O)(CR^{a}R^{b})ₘ—;
L² is —O—, —O—(CR^{a}R^{b})ₘ—, —(CR^{a}R^{b})ₘ—, —NR^{c}—(CR^{a}R^{b})ₘ—, or —S—(CR^{a}R^{b})ₘ—.
(i) X¹ is CH or CR^{c} and X² is N, or (ii) X¹ is N and X² is N, CH, or CR^{c};
each occurrence of R^{a} and R^{b} is independently hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e};
each occurrence of R^{c} is independently a hydrogen or C₁₋₆ alkyl (e.g., C₁-C₄ alkyl);
each occurrence of R^{d} and R^{e} is independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;
each occurrence of m is independently 1-4; and
p is 1 or 2.

Another embodiment is a compound of the formula (II):

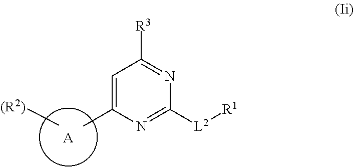

or a pharmaceutically acceptable salt thereof, wherein
each of R¹, R², R³, L² and ring A are, independently, selected from any of the embodiments described herein.

For example, in one embodiment, R³ is selected from

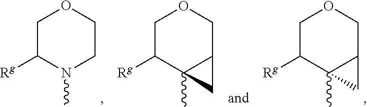

wherein each R^{g} is, independently, selected from H, substituted or unsubstituted alkyl (e.g., methyl or ethyl), OH, substituted or unsubstituted alkoxy (e.g., methoxy), halogen, CN and —NHR^{m} where R^{m} is H or substituted or unsubstituted alkyl (e.g., methyl or ethyl).

Yet another embodiment is a compound of the formula (III):

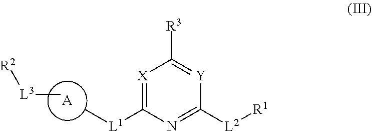

or a pharmaceutically acceptable salt thereof, wherein
each of R¹ R², R³, L¹, L², and ring A are, independently, selected from any of the embodiments described herein;
(i) X is CH or CR^{c} and Y is N, or (ii) X is N and Y is N, CH, or CR^{c}; and
L³ is —O—(CR^{a}R^{b})ₘ—, —(CR^{a}R^{b})ₘ—, —NR^{c}—(CR^{a}R^{b})ₘ—, or —S—(CR^{a}R^{b})ₘ—, —CH₂—, —CHR^{a}—, —NH—, —NR^{a}—, —C(O)—, —NHC(O)—, C(O)NH—, or a 5-membered heterocyclyl having at least one nitrogen ring atom or one oxygen ring atom, where R^{a}, R^{b}, R^{c}, and m are as defined as above (including with respect to formula (I)).

For example, in one embodiment, R³ is selected from

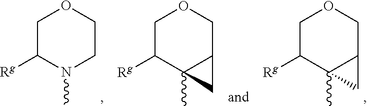

wherein each R^{g} is, independently, selected from H, substituted or unsubstituted alkyl (e.g., methyl or ethyl), OH, substituted or unsubstituted alkoxy (e.g., methoxy), halogen, CN and —NHR^{m} where R^{m} is H or substituted or unsubstituted alkyl (e.g., methyl or ethyl).

Yet another embodiment is a compound of the formula (XIII):

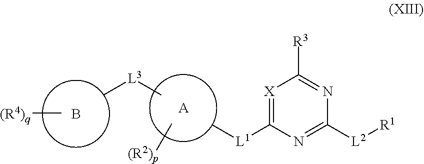

or a tautomer or pharmaceutically acceptable salt thereof, wherein
each of R¹, R², R³, L¹, L², X, and ring A are, independently, selected from any of the embodiments described herein; each occurrence of R⁴ is, independently, hydrogen, nitro, hydroxyl, halogen, cyano, substituted or unsubstituted amino (e.g., —NH₂, —NHMe, —NHMe₂), substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, substituted or unsubstituted carboxy (e.g., —COOH, —COO(C₁₋₆)alkyl), substituted or unsubstituted mercaptane, or substituted or unsubstituted heterocyclyl;
L³ is absent, optionally substituted C₁-C₇ alkylene (e.g., optionally substituted C₁-C₄ or optionally substituted C₁-C₄alkylene), —NR^{c}—, —O—, —S—, —C(O)—, —NR^{c}C(O)—, —C(O)NR^{c}—, —NR^{c}C(O)—, or —NR^{c}C(O)(CR^{a}R^{b})ₘ—;
Ring B is absent or (i) a 5 or 6-membered heteroaryl (e.g., pyridyl), a 5-6, 6-5 or 6-6 membered bicyclic heteroaryl (e.g., indole, azaindole, benzothiophene) or a heterocyclyl, each having at least one nitrogen, oxygen or sulfur ring atom, or (ii) aryl (e.g., phenyl);
each occurrence of R^{a} and R^{b} is independently hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e};
R^{c} is hydrogen or C₁₋₆ alkyl (e.g., C₁-C₄ alkyl, such as methyl);
each occurrence of R^{d} and R^{e} is independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl; and
each occurrence of m is independently 0, 1, 2, 3, 4, 5, 6 or 7 (such as 1, 2, 3, 4, 5, 6 or 7, such as 1, 2, 3 or 4, such as 1 or 2);
each occurrence of p is independently 0, 1, 2, 3 or 4, such as 0, 1 or 2; and
q is 0, 1, or 2, such as 0 or 1.

For example, in one embodiment, R³ is selected from

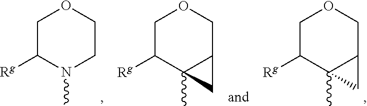

wherein each R⁹ is, independently, selected from H, substituted or unsubstituted alkyl (e.g., methyl or ethyl), OH, substituted or unsubstituted alkoxy (e.g., methoxy), halogen, CN and —NH^{m} where R^{m} is H or substituted or unsubstituted alkyl (e.g., methyl or ethyl).

Yet another embodiment is a compound of the formula (XIV):

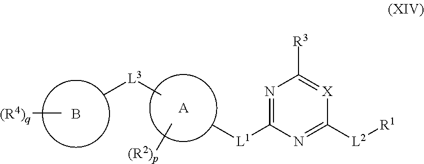

or a tautomer or pharmaceutically acceptable salt thereof,
wherein the variables (including X, R¹, R², R³, R⁴, L¹, L², L³, p, q, Ring A and Ring B) are the same as any of the embodiments described above for compounds of formula (XIII).

For example, in one embodiment, R³ is selected from

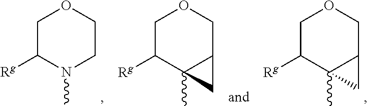

wherein each R⁹ is, independently, selected from H, substituted or unsubstituted alkyl (e.g., methyl or ethyl), OH, substituted or unsubstituted alkoxy (e.g., methoxy), halogen, CN and —NHR^{m} where R^{m} is H or substituted or unsubstituted alkyl (e.g., methyl or ethyl).

Yet another embodiment is a compound of the formula (XVI):

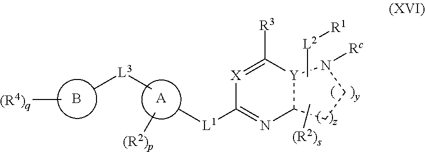

or a tautomer or pharmaceutically acceptable salt thereof, wherein
wherein variables X, R¹, R², R³, R⁴, L¹, L², L³, p, q, Ring A and Ring B are the same as any of the embodiments described above for compounds of formula (XIII);
Y is C or N;
R^{c} is absent, hydrogen or C₁₋₆ alkyl (e.g., C₁-C₄ alkyl, such as methyl);
each [symbol] is, independently, a single or double bond, with the proviso that a maximum of two [symbol] are double bonds (and adjacent [symbol] cannot both be double bonds);
y is 1, 2 or 3 (e.g., 1 or 2, such as 1);
z is 1, 2 or 3 (e.g., 1 or 2, such as 1); and
s is 0, 1, 2, 3 or 4 (e.g., 0, 1 or 2).

For example, in one embodiment, R³ is selected from

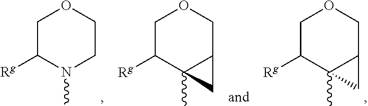

wherein each R^{g} is, independently, selected from H, substituted or unsubstituted alkyl (e.g., methyl or ethyl), OH, substituted or unsubstituted alkoxy (e.g., methoxy), halogen, CN and —NHR^{m} where R^{m} is H or substituted or unsubstituted alkyl (e.g., methyl or ethyl).

In one embodiment, the compound of formula (XVI) is of formula (XVI-A), (XVI—B) or (XVI—C).

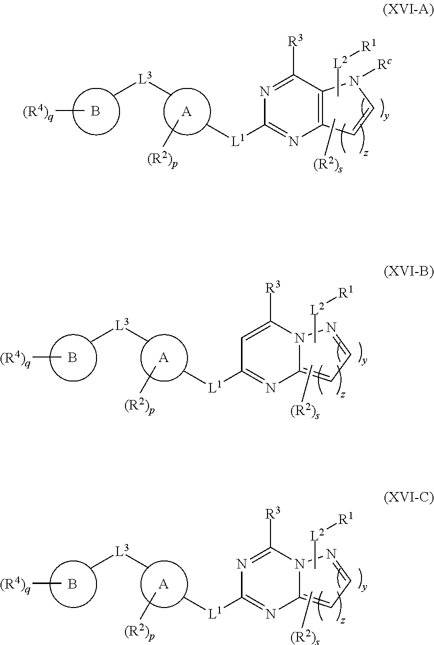

or a tautomer or pharmaceutically acceptable salt thereof, wherein the variables (including X, R¹, R², R³, R⁴, R^{c}, L¹, L², p, q, s, y, z, Ring A and Ring B) are the same as any of the embodiments described above for compounds of formula (XVI).

For example, in one embodiment, R³ is selected from

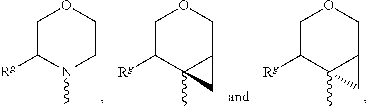

wherein each R^{g} is, independently, selected from H, substituted or unsubstituted alkyl (e.g., methyl or ethyl), OH, substituted or unsubstituted alkoxy (e.g., methoxy), halogen, CN and —NHR^{m} where R^{m} is H or substituted or unsubstituted alkyl (e.g., methyl or ethyl).

Exemplary compounds of the present include those listed below, and tautomers and pharmaceutically salts thereof.

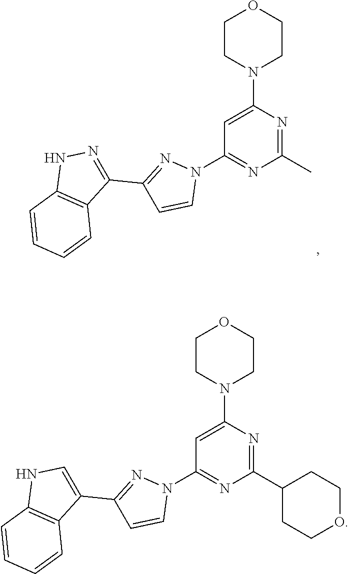 [SMILES: Cc1nc(N2CCOCC2)cc(-n2ccc(-c3n[nH]c4ccccc34)n2)n1 ; c1ccc2c(-c3ccn(-c4cc(N5CCOCC5)nc(C5CCOCC5)n4)n3)c[nH]c2c1]

Another embodiment is a method of inhibiting PIKfyve in a subject (such as a human subject) in need thereof comprising administering an effective amount of a compound of the present invention to the subject.

Yet another embodiment is a method for treating a disease or disorder associated with PIKfyve in a human subject in need thereof comprising administering an effective amount of a compound of the present invention to the subject.

Yet another embodiment is a method of treating a subject (preferably a human subject) having a neurological disease comprising administering an effective amount of a compound of the present invention to the subject. In one embodiment, the neurological disease is amyotrophic lateral sclerosis (ALS). In another embodiment, the neurological disease is frontotemporal dementia (FTD). In yet another embodiment, the neurological disease is Alzheimer's disease. In yet another embodiment, the neurological disease is Parkinson's disease. In yet another embodiment, the neurological disease is Huntington's disease. In yet another embodiment, the neurological disease is Charcot-Marie-Tooth disease (CMT).

In one embodiment, the effective amount of the compound is the amount effective to inhibit cellular PIKfyve activity in target cells in the subject. In another embodiment, the effective amount is the amount effective to induce vacuolization and disrupts intracellular trafficking in target cells.

In one embodiment, the target cell is a cancer cell. In one embodiment, the cancer cell is a lymphoma cell. In one embodiment, the lymphoma cell is a non-Hodgkin's lymphoma cell.

In one embodiment, the disease or disorder is selected from a cancer, a viral infection, or a cell proliferative disorder. For example, the cancer can be a lymphoma or melanoma. In one embodiment, the cancer is refractory or resistant to standard therapy. In one embodiment, the cancer is a non-Hodgkin's lymphoma.

One embodiment is a method of treating a viral infection in a subject in need thereof comprising administering an effective amount of a compound of the present invention to the subject. The viral infection can be caused by any type of virus such as RNA and DNA viruses. In one embodiment, the virus is Ebola virus. In another embodiment, the virus is middle east respiratory syndrome virus (MERS). In yet another embodiment, the virus is JC polyomavirus (JC). In yet another embodiment, the virus is BK polyomavirus (BK). In yet another embodiment, the virus is Herpes Simplex Virus (HSV). In yet another embodiment, the virus is Marburg virus (MarV). In yet another embodiment, the virus is Venezuelan equine encephalitis virus (VEEV). In yet another embodiment, the virus is Lymphocytic choriomeningitis virus (LCMV).

Another embodiment is method of treating Charcot-Marie-Tooth disease (CMT) in a subject, preferably a human subject, in need of such treatment, by administering an effective amount of a compound of the present invention to the subject.

One embodiment is a method for treating a lymphoma comprising administering (e.g., an effective amount of) a compound of the present invention and at least one additional active agent. In one embodiment, the at least one additional active agent is selected from ibrutinib, rituximab, doxorubicin, prednisolone, vincristine, velcade, and everolimus, and combinations thereof. In one embodiment, the method includes a therapeutic regimen comprising administering a compound of the present invention and a CHOP regimen.

In one embodiment, the method is a method for treating melanoma and the method further comprises administering at least one additional active agent to the subject in a therapeutic regimen comprising a compound of the present invention and the at least one additional active agent. In one embodiment, the at least one additional active agent is selected from dacarbazine, temozolomide, Nab-paclitaxel, carmustine, cisplatin, carboplatin, or vinblastine.

In one embodiment, the method is a method for treating a viral infection and the method further comprises administering at least one additional active agent to the subject in a therapeutic regimen comprising a compound of the present invention and the at least one additional active agent. In one embodiment, the at least one additional active agent is selected from selected from the group consisting of apilimod, APY0201, and YM-201636.

In accordance with any of the methods described herein, a compound of the present invention may also be administered in combination with a non-therapeutic agent which mitigates one or more side effects associated with the compound or increases the bioavailability of the compound. In one embodiment, the non-therapeutic agent is selected from the group consisting of ondansetron, granisetron, dolasetron and palonosetron. In another aspect, the non-therapeutic agent is selected from the group consisting of pindolol and risperidone. In another aspect, the non-therapeutic agent is selected from a cytochrome P450 3A (CYP3A) inhibitor. In one embodiment, the CYP3A inhibitor is selected from ritonavir and cobicistat.

In one embodiment, the viral infection is caused by a virus selected from the group consisting of measles, Ebola (EboV), Marburg (MarV), borna disease, and human immunodeficiency virus (HIV), severe acute respiratory system virus (SARS), and middle east respiratory syndrome virus (MERS). In one embodiment, the viral infection is caused by an EboV virus.

In one embodiment, the compound is in the form a pharmaceutical composition comprising the compound and at least one pharmaceutically acceptable carrier.

In one embodiment, the compound comprises at least 95% or at least 99% enantiomeric excess of the (R)-enantiomer. In one embodiment, the compound comprises at least 95% or at least 99% enantiomeric excess of the (S)-enantiomer.

Another embodiment is pharmaceutical composition comprising a compound of the present invention wherein the compound comprises at least 95% or at least 99% enantiomeric excess of the (R)-enantiomer or the (S)-enantiomer.

### DETAILED DESCRIPTION OF THE INVENTION

### Definitions

As used herein the following definitions shall apply unless otherwise indicated. Further, many of the groups defined herein can be optionally substituted. The listing of substituents in the definition is exemplary and is not to be construed to limit the substituents defined elsewhere in the specification.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₆ alkyl” refers to an alkyl group as defined above having up to 6 carbon atoms. The term “C₁₋₃ alkyl” refers to an alkyl group as defined above having up to 3 carbon atoms.

In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “C₂₋₆ alkenyl” refers to an alkenyl group as defined above having up to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butnyl. The term “C₂₋₆ alkynyl” refers to an alkynyl group as defined above having up to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined above attached via an oxygen linkage to the rest of the molecule. The term “substituted alkoxy” refers to an alkoxy group where the alkyl constituent is substituted (i.e., —O-(substituted alkyl). For example “alkoxy” refers to the group —O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and sprirobicyclic groups, e.g., spiro[4.4]non-2-yl. The term “C₃₋₆ cycloalkyl” refers to a cycloalkyl group as defined above having up to 6 carbon atoms.

The term “cycloalkylalkyl”, unless otherwise specified, refers to a cyclic ring-containing radical containing in the range of about 3 up to 8 carbon atoms directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 up to 8 carbon atoms with at least one carbon-carbon double bond such as cyclopropenyl, cyclobutenyl, and cyclopentenyl. The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 up to 20 carbon atoms such as phenyl, naphthyl, tetrahydronaphthyl, indanyl, and biphenyl.

The term “arylalkyl”, unless otherwise specified, refers to an aryl group as defined above directly bonded to an alkyl group as defined above, e.g., —CH₂C₆H₅ and —C₂H₅C₆H₅.

The term “heterocyclic ring”, unless otherwise specified, refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom.

The term “heterocyclyl”, unless otherwise specified, refers to a heterocylic ring radical as defined above. The heterocylcyl ring radical may be attached to the main structure at any heteroatom or carbon ring atom. In appropriate circumstances, the term “heterocyclyl” refers to a hydrocarbon chain radical as mentioned above which is bivalent.

The term “heterocyclylalkyl”, unless otherwise specified, refers to a heterocylic ring radical as defined above directly bonded to an alkyl group. The heterocyclylalkyl radical may be attached to the main structure at any carbon atom in the alkyl group. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl”, unless otherwise specified, refers to an optionally substituted 5 to 14 member aromatic ring having one or more heteroatoms selected from N, O, and S as ring atoms. The heteroaryl may be a mono-, bi- or tricyclic ring system. Examples of such “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamo holinyl, thiamorpholinyl sulfoxide, thiamo holinyl sulfone, dioxaphospholanyl, oxadiazolyl, chromanyl, and isochromanyl.

The term “5 or 6-membered heteroaryl” refers to a heteroaryl having 5- or 6-ring atoms. The term “5-6 or 6-5 membered bicyclic heteroaryl” refers to a bicyclic heteroaryl with a five-membered ring fused to a six-membered ring, where the 5-membered ring is bound to the rest of the molecule (referred as a “5-6 membered bicyclic heteroaryl”) or the 6-membered ring is bound to the rest of the molecule (referred as a “6-5 membered bicyclic heteroaryl”). The term “6-6 membered bicyclic heteroaryl” refers to a bicyclic heteroaryl with a six-membered ring fused to a another six-membered ring, where one of the 6-membered rings is bound to the rest of the molecule.

The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a heteroaryl ring radical as defined above directly bonded to an alkyl group. The heteroarylalkyl radical may be attached to the main structure at any carbon atom from alkyl group.

The term “cyclic ring” refers to a cyclic ring containing 3 to 10 carbon atoms.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, ═N—NR^{x}R^{y}, —NR^{x}C(O)OR^{y}, —NR^{x}R^{y}, —NR^{x}C(O)R^{y}, —NR^{x}C(S)R^{y}, —NR^{x}C(S)NR^{y}R^{z}, —SONR^{x}R^{y}, —SO₂NR^{x}R^{y}, —OR^{x}, —OR^{x}C(O)NR^{y}R^{z}, —OR^{x}C(O)OR^{y}, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, and —ONO₂, wherein R^{x}, R^{y} and R^{z} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} (e.g., R^{x} can be hydrogen or C₁₋₆ alkyl) or S. Substitution or the combinations of substituents envisioned by this invention are preferably those that result in the formation of a stable or chemically feasible compound. The term stable as used herein refers to the compounds or the structure that are not substantially altered when subjected to conditions to allow for their production, detection and preferably their recovery, purification and incorporation into a pharmaceutical composition. The substituents in the aforementioned “substituted” groups cannot be further substituted. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, tert-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

A “leaving group or atom” is any group or atom that will, under the reaction conditions, cleave from the starting material, thus promoting reaction at a specified site. Suitable examples of such groups unless otherwise specified are halogen atoms and mesyloxy, p-nitrobenzensulphonyloxy and tosyloxy groups.

The term “prodrug” refers to a compound, which is an inactive precursor of a compound, converted into its active form in the body by normal metabolic processes. Prodrug design is discussed generally in Hardma, et al. (Eds.), Goodman and Gilman's The Pharmacological Basis of Therapeutics, 9th ed., pp. 11-16 (1996). A thorough discussion is provided in Higuchi, et al., Prodrugs as Novel Delivery Systems, Vol. 14, ASCD Symposium Series, and in Roche (ed.), Bioreversible Carriers in Drug Design, American Pharmaceutical Association and Pergamon Press (1987). To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into circulation, thereby effectively increasing the biological half-life of the originally administered compound.

Additionally, the instant invention also includes the compounds which differ only in the presence of one or more isotopically enriched atoms for example replacement of hydrogen with deuterium or tritium, the replacement of a carbon by ¹³C- or ¹⁴C-enriched carbon, or the replacement of a nitrogen by ¹⁵N.

The compounds of the present invention may also contain unnatural proportions of atomic isotopes at one or more of atoms that constitute such compounds. For example, the compounds may be radiolabeled with radioactive isotopes, such as for example tritium, iodine-125 (¹²⁵I) or carbon-14 (¹⁴C). All isotopic variations of the compounds of the present invention, whether radioactive or not, are encompassed within the scope of the present invention.

Pharmaceutically acceptable salts forming part of this invention include salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, and thiamine; chiral bases such as alkylphenylamine, glycinol, and phenyl glycinol; salts of natural amino acids such as glycine, alanine, valine, leucine, isoleucine, norleucine, tyrosine, cystine, cysteine, methionine, proline, hydroxy proline, histidine, ornithine, lysine, arginine, and serine; quaternary ammonium salts of the compounds of invention with alkyl halides, and alkyl sulphates. Salts may include acid addition salts where appropriate which are sulphates, nitrates, phosphates, perchlorates, borates, hydrohalides (e.g., hydrochlorides), acetates, tartrates, maleates, citrates, fumarates, succinates, palmoates, methanesulphonates, benzoates, salicylates, benzenesulfonates, ascorbates, glycerophosphates, and ketoglutarates. Salts can be formed by methods known in the art.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. In one embodiment, the amount of compound administered ranges from about 0.1 mg to 5 g, from about 1 mg to 2.0 g, from about 100 mg to 1.5 g, from about 200 mg to 1.5 g, from about 400 mg to 1.5 g, and from about 400 mg to 1.0 g.

As used herein, the term “treating” refers to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

A “neurological disease” is any disease that causes electrical, biochemical, or structural abnormalities in the brain, spine, or neurons. For example, a neurological disease may be a neurodegenerative disease. The neurodegenerative disease may result in motor neuron degeneration, for example. The neurological disease may be amyloid lateral sclerosis, Huntington's disease, Alzheimer's disease, or frontotemporal dementia, for example. Further examples of neurological diseases include, but are not limited to, Parkinson's disease, multiple sclerosis, peripheral myopathy, Rasmussen's encephalitis, attention deficit hyperactivity disorder, autism, central pain syndromes, anxiety, and/or depression, for example.

Neurodegenerative diseases result in the progressive destruction of neurons that affects neuronal signaling. For example, a neurodegeneration may be amyotrophic lateral sclerosis, Alzheimer's disease, Huntington's disease, Friedreich's ataxia, Lewy body disease, Parkinson's disease, spinal muscle atrophy, primary lateral sclerosis, progressive muscle atrophy, progressive bulbar palsy, and pseudobulbar palsy.

The term “pharmaceutically acceptable excipient” includes, but is not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants/flavoring, carriers, buffers, stabilizers, solubilizers, and combinations thereof. Except insofar as any conventional media or agent is incompatible with the active ingredient, its use in the therapeutic compositions of the invention is contemplated. Supplementary active ingredients can also be incorporated into the compositions.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the mammal is a human. The term “patient” refers to a human subject.

In accordance with the methods described herein, a “subject in need of” is a subject having a disease, disorder or condition, or a subject having an increased risk of developing a disease, disorder or condition relative to the population at large. The subject in need thereof can be one that is “non-responsive” or “refractory” to a currently available therapy for the disease or disorder, for example cancer. In this context, the terms “non-responsive” and “refractory” refer to the subject's response to therapy as not clinically adequate to relieve one or more symptoms associated with the disease or disorder. In one aspect of the methods described here, the subject in need thereof is a subject having cancer whose cancer is refractory to standard therapy or whose cancer has recurred following standard treatment.

### Pharmaceutical Compositions

One embodiment is a pharmaceutical composition suitable for use in a subject, such as a human. The pharmaceutical composition may comprise at least one pharmaceutically acceptable excipient or carrier.

The pharmaceutical composition may also include at least one additional active agent, such as an alkylating agent, an intercalating agent, a tubulin binding agent, a corticosteroid, or any combination of any of the foregoing. Examples of additional active agents include, but are not limited to, ibrutinib, rituximab, doxorubicin, prednisolone, vincristine, velcade, and everolimus, In one embodiment, the at least one additional active agent is a therapeutic agent selected from cyclophosphamide, hydroxydaunorubicin (also referred to as doxorubicin) vincristine, prednisone, prednisolone, and any combination of any of the foregoing.

The pharmaceutical composition may include one or more non-therapeutic agents, such as ondansetron, granisetron, dolasetron, palonosetron, pindolol, risperidone, or any combination of any of the foregoing.

A pharmaceutical composition can be provided as a dosage unit form, such as an ampoule, a vial, a suppository, a dragee, a tablet, or a capsule.

The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration, in the form of a sterile aqueous solution or dispersion suitable for administration by either direct injection or by addition to sterile infusion fluids for intravenous infusion, or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

### Preparation of the Compounds

The compounds described herein may be prepared as described in International Publication No. WO 2021/163727, which is hereby incorporated by reference.

As an example, some of the compounds of the present invention may be prepared as follows.

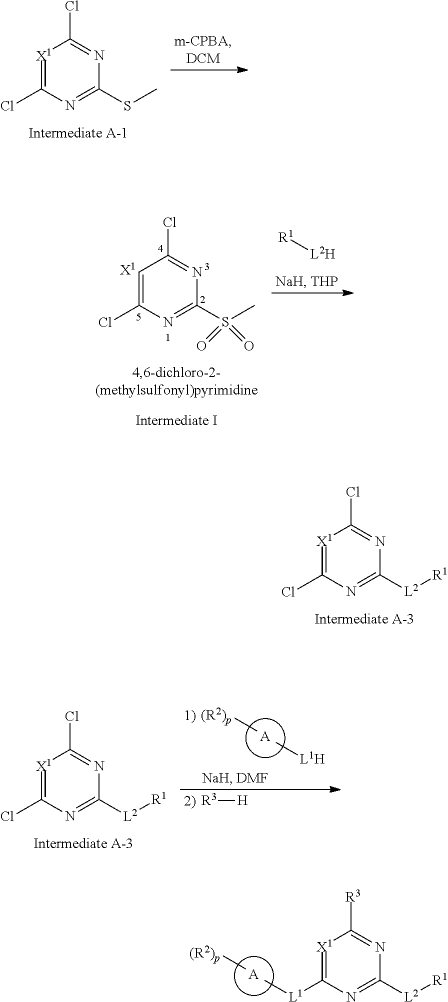

Starting intermediate A-1 is oxidized, for example by reaction with m-CPBA (meta-chloroperoxybenzoic acid) in a solvent, such as dichloromethane, to produce intermediate I. Intermediate I is then reacted with R¹-L²H, for example, in the presence of a base (such as NaH) and in a solvent, such as THF, to form Intermediate A-3. Intermediate A-3 is first reacted with

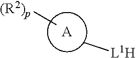

and then with R³—H to form the final compound. The other compounds of the present invention can be prepared in an analogous manner.

### Methods of Treatment

The compounds of the present invention are useful as PIKfyve kinase inhibitors.

One embodiment a method of treating a subject having a neurological disease comprising administering to the subject an effective amount of a compound of the present invention (or a pharmaceutical composition of the present invention) to the subject. In a preferred embodiment, the subject is a human subject. The PIKfyve kinase inhibitors described herein may be used in the methods for treating a neurological disease described in U.S. Patent Publication No. 2018/0161335, which is hereby incorporated by reference in its entirety. For example, the neurological disease may be one that has neuronal death generated by intracellular aggregates.

In certain embodiments, the method includes treating amyotrophic lateral sclerosis (ALS). In certain embodiments, the method includes treating frontotemporal dementia (FTD). In certain embodiments, the method includes treating a neurological disease that is associated with aberrant endosomal trafficking. In certain embodiments, the method includes treating a neurological disease that is associated with aberrant lysosomal trafficking. In further embodiments, the method includes treating a subject who has a (GGGGCC)ₙ repeat expansion in the C9ORF72 gene. In further embodiments, the subject is haploinsufficient for C9ORF72. In further embodiments, the method includes treating patients who have a 50% or greater reduction in C9ORF72 protein activity. In further embodiments, the method includes a C9ORF72 gene product that comprises a dipeptide repeat resulting from the (GGGGCC)ₙ expansion. In further embodiments, the method includes a gain-of-function or loss of function mutation resulting from the (GGGGCC)ₙ expansion. In further embodiments, the neurological disease is associated with neuronal hyperexcitability.

One embodiment is a method of treating a subject having amyotrophic lateral sclerosis (ALS) comprising administering to the subject (preferably a human subject) an effective amount of a compound of the present invention. Another embodiment is a method of treating a subject having frontotemporal dementia (FTD) comprising administering to the subject (preferably a human subject) an effective amount of a compound of the present invention. Yet another embodiment is a method of treating a subject having Alzheimer's disease comprising administering to the subject (preferably a human subject) an effective amount of a compound of the present invention. Yet another embodiment is a method of treating a subject having Parkinson's disease comprising administering to the subject (preferably a human subject) an effective amount of a compound of the present invention. Yet another embodiment is a method of treating a subject having Huntington's disease comprising administering to the subject (preferably a human subject) an effective amount of a compound of the present invention. Yet another embodiment is a method of treating a subject having Charcot-Marie-Tooth disease (CMT) comprising administering to the subject (preferably a human subject) an effective amount of a compound of the present invention.

The method may further comprise also administering an effective amount of a potassium channel activator, an inhibitor of a glutamate receptor (such as the receptor NMDA, AMPA, or kainite) (e.g., AP5, CNQX, and NBQX), or any combination of any of the foregoing.

PIKfyve is a phosphoinositide kinase (PIK) that contains a FYVE-type zinc finger domain, which binds phosphatidylinositol 3-phosphate (PI3P). PIKfyve phosphorylates PUP to produce PI(3,5)P₂, which is involved in cellular processes including membrane trafficking and cytoskeletal reorganization. The inhibition of PIKfyve by a compound described herein is useful in treating not only cancer, but also Charcot-Marie-Tooth disease and certain viral infections, such as those caused by a virus selected from measles, Ebola virus (EboV), Marburg virus (MarV), borna disease, and human immunodeficiency virus (HIV), severe acute respiratory system virus (SARS), middle east respiratory syndrome virus (MERS), JC polyomavirus (JC), BK polyomavirus (BK), Herpes Simplex Virus (HSV), Venezuelan equine encephalitis virus (VEEV) and Lymphocytic choriomeningitis virus (LCMV). The viral infection can be caused by any type of virus such as RNA and DNA viruses.

One embodiment is a method of treating a viral infection in a subject in need thereof comprising administering an effective amount of a compound of the present invention to the subject. In one embodiment, the virus is Ebola virus. In another embodiment, the virus is middle east respiratory syndrome virus (MERS). In yet another embodiment, the virus is JC polyomavirus (JC). In yet another embodiment, the virus is BK polyomavirus (BK). In yet another embodiment, the virus is Herpes Simplex Virus (HSV). In yet another embodiment, the virus is Marburg virus (MarV). In yet another embodiment, the virus is Venezuelan equine encephalitis virus (VEEV). In yet another embodiment, the virus is Lymphocytic choriomeningitis virus (LCMV).

One embodiment is a method for treating a cell proliferative disease, a cancer, or a viral infection in a subject, preferably a human subject, in need of such treatment, by administering an effective amount of a compound of the present invention or a pharmaceutical composition comprising the same, to the subject.

The compounds described herein are useful for treating cancer. In one embodiment, the cancer is brain cancer, glioma, sarcoma, breast cancer, lung cancer, non-small-cell lung cancer, mesothelioma, appendiceal cancer, genitourinary cancers, renal cell carcinoma, prostate cancer, bladder cancer, testicular cancer, penile cancer, cervical cancer, ovarian cancer, von Hippel Lindau disease, head and neck cancer, gastrointestinal cancer, hepatocellular carcinoma, gallbladder cancer, esophageal cancer, gastric cancer, colorectal cancer, pancreatic cancer, neuroendocrine tumors, thyroid tumor, pituitary tumor, adrenal tumor, hematological malignancy, or leukemia. In another embodiment, the cancer is B cell lymphoma. In another embodiment, the cancer is a melanoma.

In one embodiment the cancer is a lymphoma. In one embodiment, the lymphoma is a B cell lymphoma. In one embodiment, the B cell lymphoma is selected from the group consisting of a Hodgkin's B cell lymphoma and a non-Hodgkin's B cell lymphoma. In one embodiment, the B cell lymphoma is a non-Hodgkin's B cell lymphoma selected from the group consisting of DLBCL, follicular lymphoma, marginal zone lymphoma (MZL) or mucosa associated lymphatic tissue lymphoma (MALT), small cell lymphocytic lymphoma (overlaps with chronic lymphocytic leukemia) and mantle cell lymphoma. In one embodiment, the B cell lymphoma is a non-Hodgkin's B cell lymphoma selected from the group consisting of Burkitt's lymphoma, Primary mediastinal (thymic) large B-cell lymphoma, Lymphoplasmacytic lymphoma, which may manifest as Waldenstrom macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma, Primary central nervous system lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Intravascular large B-cell lymphoma, ALK-positive large B-cell lymphoma, and Plasmablastic lymphoma.

In one embodiment, the method is a method of treating a lymphoma using a combination therapy comprising a compound of the present invention and a chemotherapy regimen for the treatment of the lymphoma. In one embodiment, the chemotherapy regimen is the CHOP regimen. In another embodiment, the chemotherapy regimen is selected from COOP, CVP, EPOCH, Hyper-CVAD, ICE, R—CHOP, and R—CVP.

In the methods described here, the compounds can be administered by any suitable route, such as an oral, intravenous, or subcutaneous route.

All references cited herein are incorporated by reference.

What is claimed is:

1. A compound of the formula (I) 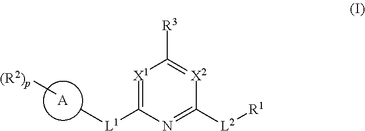
or a pharmaceutically acceptable salt thereof, wherein
R¹ is hydroxy, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;
each occurrence of R² is independently substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl;
R³ is selected from 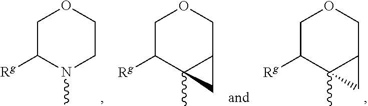
each R⁹ is, independently, selected from H, substituted or unsubstituted alkyl, OH, substituted or unsubstituted alkoxy, halogen, CN and —NHR^{m} where R^{m} is H or substituted or unsubstituted alkyl;
Ring A is (i) a 5 or 6-membered heteroaryl, a 5-6, 6-5 or 6-6 membered bicyclic heteroaryl, or a heterocyclyl, each having at least one nitrogen or oxygen ring atom, or (ii) phenyl;
L¹ is absent, C₁-C₂ alkylene, —NR^{c}—, —O—, —S—, —C(O)—, —NHC(O)—, —C(O)NH—, —NR^{c}C(O)—, or —NR^{c}C(O)(CR^{a}R^{b})—;
L² is absent, —O—, —O—(CR^{a}R^{b})ₘ—, —(CR^{a}R^{b})ₘ—, —NR^{c}—(CR^{a}R^{b})ₘ—, or —S—(CR^{a}R^{b})ₘ—;
(i) X¹ is CH or CR^{c} and X² is N, or (ii) X¹ is N and X² is N, CH, or CR^{c};
each occurrence of R^{a} and R^{b} is independently hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e};
each occurrence of R^{c} is independently a hydrogen or C₁₋₆ alkyl (e.g., C₁-C₄ alkyl);
each occurrence of R^{d} and R^{e} is independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;
each occurrence of m is independently 1-4; and
p is 1 or 2,
wherein when R³ is 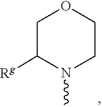
R^{g} is not hydrogen.

2. The compound of claim 1, wherein X¹ is CH, X² is N, L¹ is absent, and L² is absent.

3. The compound of claim 1 or 2, wherein R¹ is heterocyclyl or heteroaryl.

4. The compound of claim 1 or 2, wherein R¹ is be selected from (the squiggly lines indicate the point of attached to the rest of the molecule)

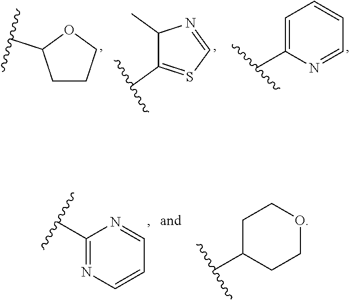

5. The compound of claim 1 or 2, wherein R¹ is methyl.

6. The compound of claim 1 or 2, wherein each occurrence of R¹ is independently selected from

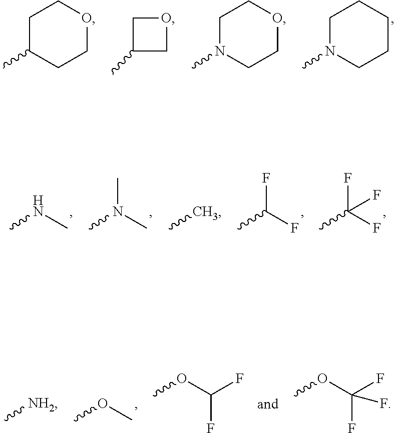

7. The compound of claim 6, wherein L² is absent.

8. The compound of claim 1 or 2, wherein R¹ is selected from

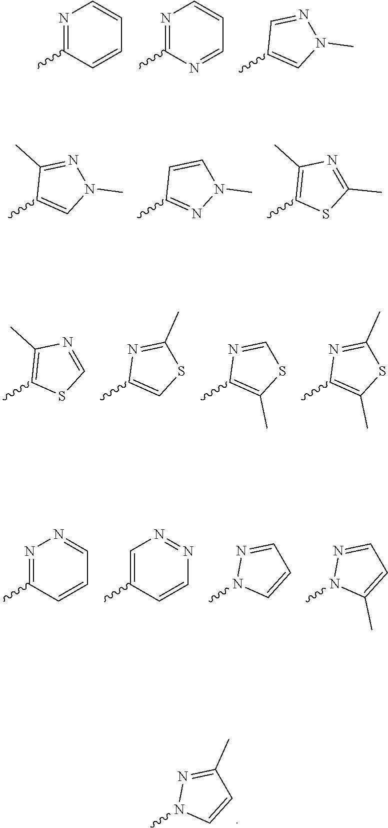

9. The compound of claim 8, wherein L² is —O—, —O—(CR^{a}R^{b})ₘ—, —(CR^{a}R^{b})ₘ—, —NR^{c}—(CR^{a}R^{b})ₘ—, or —S—(CR^{a}R^{b})ₘ—.

10. The compound of any one of the preceding claims, wherein each occurrence of R² is independently selected from 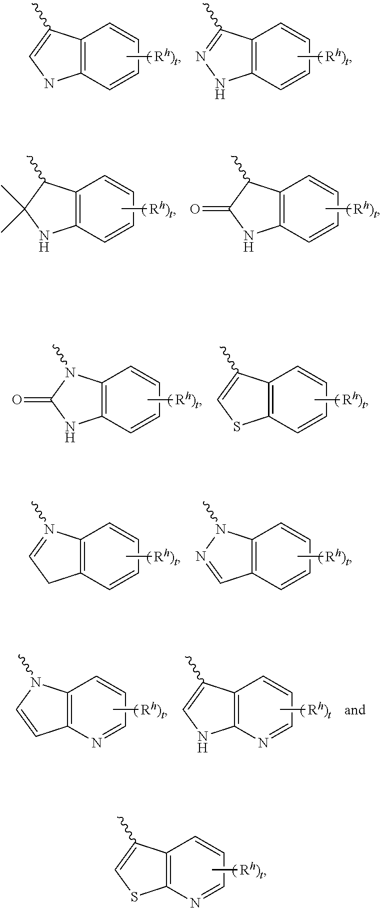
wherein
each occurrence of R^{h} is, independently H, halogen, substituted or unsubstituted alkyl, substituted or unsubstituted alkoxy, —CN, —NH₂, —NHR^{m} or —NR^{m}₂, or two R^{h} are joined to form a saturated or unsaturated 5- to 7-membered ring;
each R^{m} is, independently, H or substituted or unsubstituted alkyl; and
t is 0, 1, or 2.

11. The compound of any one of the preceding claims, wherein ring A is selected from 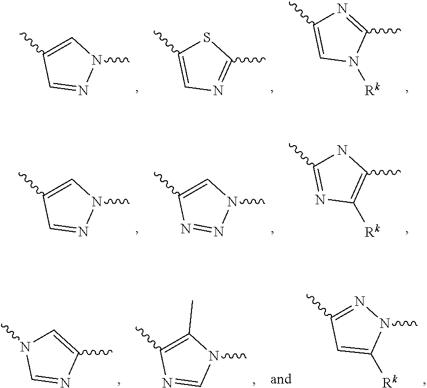
wherein
each R^{k} is, independently, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl,
each squiggly line ( [symbol] ) on the righthand side of ring A represents the point of attachment of ring A to variable L¹, and
each squiggly line on the left-hand side of ring A represents the point of attachment of ring A to variable R².

12. The compound of any one of the preceding claims, wherein L¹ is absent.

13. The compound of any one of claims 1-6 and 8-11, wherein L¹ is —NH—, —N(CH₃)—, —O—, or —CH₂—.

14. The compound of any one of the preceding claims, wherein L² is absent.

15. The compound of any one of claims 1-13, wherein L² is —OCH₂CH₂—, —OCH₂—, or —OCH₂CH₂CH(OH)CH₂—.

16. The compound of any one of claims 1-13, wherein L² is —(CR^{a}R^{b})ₘ—.

17. The compound of any one of claims 1-13, wherein -L²-R¹ is —OCH₂CH₂CH(OH)CH₂OH.

18. The compound of any one of the preceding claims, wherein X¹ is CH.

19. The compound of any one of the preceding claims, wherein each occurrence of R^{a} and R^{b} is independently hydrogen, hydroxy, or hydroxy(C₁₋₄)alkyl.

20. The compound of any one of the preceding claims, wherein each occurrence of R^{a} and R^{b} is independently hydrogen or hydroxy.

21. The compound of any one of the preceding claims, wherein m is 1.

22. The compound of any one of claims 1-20, wherein m is 2.

23. The compound of any one of the preceding claims, wherein p is 1.

24. The compound of any one of claims 1-22, wherein p is 2.

25. The compound of any of the preceding claims, wherein the moiety 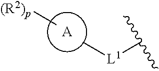
is selected from 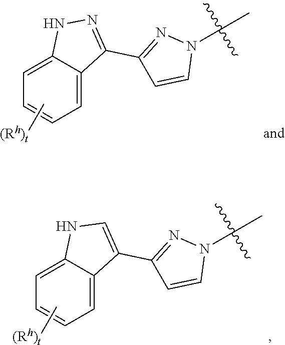
wherein
each occurrence of R^{h} is, independently H, halogen, substituted or unsubstituted alkyl (e.g., methyl or ethyl), substituted or unsubstituted alkoxy (e.g., methoxy or ethoxy), —CN, —NH₂, —NHR^{m} or —NR^{m}₂, or two R^{h} are joined to form a saturated or unsaturated 5- to 7-membered ring;
each R^{m} is, independently, R^{m} is H or substituted or unsubstituted alkyl (e.g., methyl or ethyl); and
t is 0, 1, or 2.

26. A compound selected from 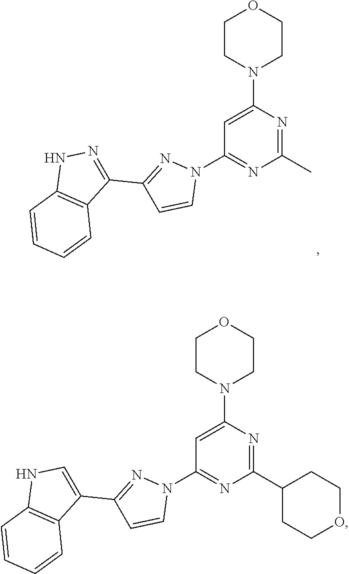 [SMILES: Cc1nc(N2CCOCC2)cc(-n2ccc(-c3n[nH]c4ccccc34)n2)n1 ; c1ccc2c(-c3ccn(-c4cc(N5CCOCC5)nc(C5CCOCC5)n4)n3)c[nH]c2c1]
and pharmaceutically acceptable salts thereof.

27. A method of inhibiting PIKfyve in a subject in need thereof comprising administering an effective amount of a compound of any one of claims 1-26.

28. A method for treating a disease or disorder associated with PIKfyve in a human subject in need thereof comprising administering an effective amount of a compound of any one of claims 1-26.

29. A method of treating a subject having a neurological disease comprising administering to the subject an effective amount of a compound of any one of claims 1-26.

30. The method of claim 29, wherein the neurological disease is amyotrophic lateral sclerosis (ALS).

31. The method of claim 29, wherein the neurological disease is frontotemporal dementia (FTD).

32. The method of claim 29, wherein the neurological disease is Alzheimer's disease.

33. The method of claim 29, wherein the neurological disease is Parkinson's disease.

34. The method of claim 29, wherein the neurological disease is Huntington's disease.

35. The method of claim 29, wherein the neurological disease is Charcot-Marie-Tooth disease (CMT).

36. A method of treating a viral infection in a subject in need thereof comprising administering an effective amount of a compound of any one of claims 1-26.

37. The method of claim 36, wherein the virus is Ebola virus.

38. The method of claim 36, wherein the virus is middle east respiratory syndrome virus (MERS).

39. The method of claim 36, wherein the virus is JC polyomavirus (JC).

40. The method of claim 36, wherein the virus is BK polyomavirus (BK).

41. The method of claim 36, wherein the virus is Herpes Simplex Virus (HSV).

42. The method of claim 36, wherein the virus is Marburg virus (MarV).

43. The method of claim 36, wherein the virus is Venezuelan equine encephalitis virus (VEEV).

44. The method of claim 36, wherein the virus is Lymphocytic choriomeningitis virus (LCMV).
